(19) United States
(12) Patent Application Publication
(10) Pub. No.: US 2007/0141713 A1
(43) Pub. Date: Jun. 21, 2007

(54) METHOD OF DIAGNOSING CHRONIC DIARRHEA

(76) Inventor: Thomas M. Donndelinger, Nampa, ID (US)

Correspondence Address: KIRTON AND MCCONKIE, 60 EAST SOUTH TEMPLE,, SUITE 1800, SALT LAKE CITY, UT, 84111, US

(21) Appl. No.: 11/613,717

(22) Filed: Dec. 20, 2006

Related U.S. Application Data
(60) Provisional application No. 60/752,618, filed on Dec. 21, 2005.

(51) Int. Cl. G01N 33/48

(52) U.S. Cl. 436/63; 436/173

(57) ABSTRACT

A method for obtaining information about a chronic diarrhea condition in a patient, including possible diagnosis of chronic diarrhea, is disclosed. In the method, a sample of a gastrointestinal secretion that contains mitochondria is obtaining from the patient. The sample is analyzed using an analytical technique, such as mass spectroscopy, to obtain a unique proteomic pattern characteristic of the proteins within the secretion. The proteomic pattern of the sample is compared with a proteomic pattern profile database to determine differences and similarities of the proteomic pattern to the proteomic pattern profile database. The proteomic pattern database may include profiles of normal and/or abnormal proteomic patterns.

### CROSS-REFERENCE TO RELATED APPLICATION

This application claims the benefit of U.S. Provisional Patent Application 60/752,618, filed Dec. 21, 2005.

### BACKGROUND OF THE INVENTION

Many people suffer from the medical problem of chronic diarrhea. Chronic diarrhea is defined as diarrhea that lasts more than two weeks. This problem is usually painless and persistent and is normally not accompanied by more serious conditions such as bleeding, anemia, weight loss or fatigue. However, this condition is a nuisance and an embarrassment to many people. Accordingly, doctors have, for years, looked for causes and ways to treat chronic diarrhea.

There are many different causes of chronic diarrhea. For example, some types of chronic diarrhea are caused by an infection such as would be caused by a parasite, a bacterial infection or a viral infection. Other cases of chronic diarrhea are caused by medications, antibiotics, food additives, or other drugs ingested into the body. Still other cases of chronic diarrhea are caused by more serious medical conditions such as tumors, diabetes, thyroid and other endocrine diseases, food allergies, reduced blood flow into the intestine, colitis, and/or Crohn's disease. Previous surgery or radiation treatments to the abdomen or gastrointestinal tract may also be the source of some forms of chronic diarrhea.

Yet, despite the knowledge regarding chronic diarrhea, there are some cases of chronic diarrhea for which there are no known causes. In fact, it is estimated that twenty (20) to thirty (30) percent of all cases of chronic diarrhea have no known cause. Despite numerous tests and analyses, doctors simply cannot pinpoint why the patient suffers from chronic diarrhea.

Some doctors, upon realizing that they do not know the cause of the patient's diarrhea, will advise patients that the diarrhea problem is caused by stress, emotional problems, psychological problems, etc. Accordingly, these doctors will insist that the patients change their lifestyle—by reducing stress, seeking professional counseling, etc.—as a means of coping with the chronic diarrhea. Of course, such “treatments” are often difficult to implement, expensive, drastic, and ineffective and are thus disfavored.

Moreover, most of the tests that are currently used to diagnose diarrhea require multiple analyses by the doctor. Not only will multiple tests require the patients to repeatedly visit the doctor, but more importantly, some of the tests required to diagnose chronic diarrhea are uncomfortable, embarrassing, and unpleasant.

Accordingly, it would be an advancement in the art to provide a new method which will be easy to administer and reliable. Moreover, this new method should also preferably provide the doctor with additional information regarding the cause of the chronic diarrhea. Such a method for diagnosing chronic diarrhea is disclosed herein.

### BRIEF SUMMARY OF THE INVENTION

The present invention is directed to a method for diagnosing or obtaining information about a chronic diarrhea condition in a patient based upon proteomic patterns in gastrointestinal secretions. The invention is based upon the principles that humans secrete mitochondria rich cell fragments in an apocrine mechanism along with the central mucous into their gastrointestinal tract and that mitochondria and cytoplasm contain unique proteomic information. These mitochondria are usually accompanied by some cytoplasm, cell membranes, and mucous. Although not being exclusively bound by this theory, mitochondria are secreted in the gastrointestinal tract to react with oxygen, thereby rendering the gastrointestinal tract an anaerobic environment.

The method of the present embodiments involves obtaining a sample of gastrointestinal secretions containing mitochondria, mucous, cytoplasm, etc. from the patient. Generally, this sample of gastrointestinal secretions will be obtained as the patient is undergoing endoscopy. Endoscopy is currently one of the more commonly used tests performed on patients with chronic diarrhea, and as such, patients will generally not be subjected to another test in order to accomplish the present method.

Endoscopy is usually performed on a patient after the patient's bowel and gastrointestinal system has been purged. Accordingly, if gastrointestinal secretions are collected from the patient during endoscopy, these secretions will likely be substantially clean and free of excrement or other impurities. As such, little purification of these gastrointestinal secretions will likely be necessary in order to perform the present method.

If the gastrointestinal secretions are prepared as a tissue slide, conventional laser capture techniques may be used to isolate and obtain a specific portion of the sample for further analysis, including, but not limited to, biochemical analysis, PCR testing, Raman spectroscopy chromatography including two-dimensional gel chromatography, GC-MS, GC-MS/MS, etc.

Once the sample of gastrointestinal secretions has been obtained, the sample may be analyzed using mass spectroscopy. In many embodiments, gas chromatography-mass spectroscopy (GC-MS) will be used. In some cases gas chromatography-mass spectroscopy mass spectroscopy (GC-MS/MS) in tandem will be used. In other embodiments, surface-enhanced laser desorption ionization time-of-flight (“SELDI-TOF”) or matrix-assisted laser desorption ionization time-of-flight (“MALDI-TOF”) mass spectroscopy will be used. Persons skilled in the art will appreciate that other known and novel analytical methods may be used to analyze the gastrointestinal secretions.

As is known in the art, the mass spectroscopy of the sample of the gastrointestinal secretion will produce a mass chromatogram having various peaks. The gastrointestinal secretions contain proteins and genetic information from the mitochondria, cytoplasm, cell membranes, mucous (and any other compounds). These components of the secretions may be separated by ultracentrifugation, if desired. The mass spectroscopy results of the gastrointestinal secretions include a spectrum or “fingerprint” of expressed genetic information relating to the gastrointestinal tract. More specifically, the mass spectroscopy results provide a proteomic pattern for the proteome of the mitochondria in the gastrointestinal secretions and the proteome of the cytoplasm in the secretions. If the components of the secretions are separated first, then separate mass spectroscopy analysis may occur which will provide a specific proteomic pattern for each component.

Once the results of the mass spectroscopy from the sample of the gastrointestinal secretions have been obtained, these results will then be analyzed and compared to a database of mass spectrographic results. This database of mass spectrographic results is created by taking numerous proteomic patterns from numerous patients and/or healthy volunteers to create profiles of “normal” and “abnormal” gastrointestinal secretions. The database samples will include patients who are known to have chronic diarrhea as well as from patients who do not have chronic diarrhea. If appropriate, the abnormal gastrointestinal secretions will be further subset into the various types of chronic diarrhea mentioned earlier and other known gastrointestinal disorders as well as the group of chronic diarrhea types that have heretofore been undiagnosable.

Generally, the proteomic pattern diagnostics used herein compares the mass spectrographic proteomic patterns to the proteomic pattern profiles of the database. Adaptive artificial intelligence bioinformatics can be used to read the pathologic states reflected in the proteomic pattern. Bioinformatic pattern recognition tools can recognize complex data from thousands of proteins simultaneously. This information may indicate that the patient has chronic diarrhea and provide diagnostic information about the type or cause of the condition.

### BRIEF DESCRIPTION OF THE SEVERAL VIEWS OF THE DRAWINGS

FIG. 1 is a photograph of a mitochondria rich secretion from a colon mucosal crypt biopsy in which the mitochondria rich secretion is indicated with arrows.

FIG. 2 shows mitochondria in apocrine secretions accompanying mucus secretions in the colon crypt lumen, wherein the presence of mitochondria is verified through the use of a mitochondria-specific Mito Tracker fluorescent stains.

### DETAILED DESCRIPTION OF THE INVENTION

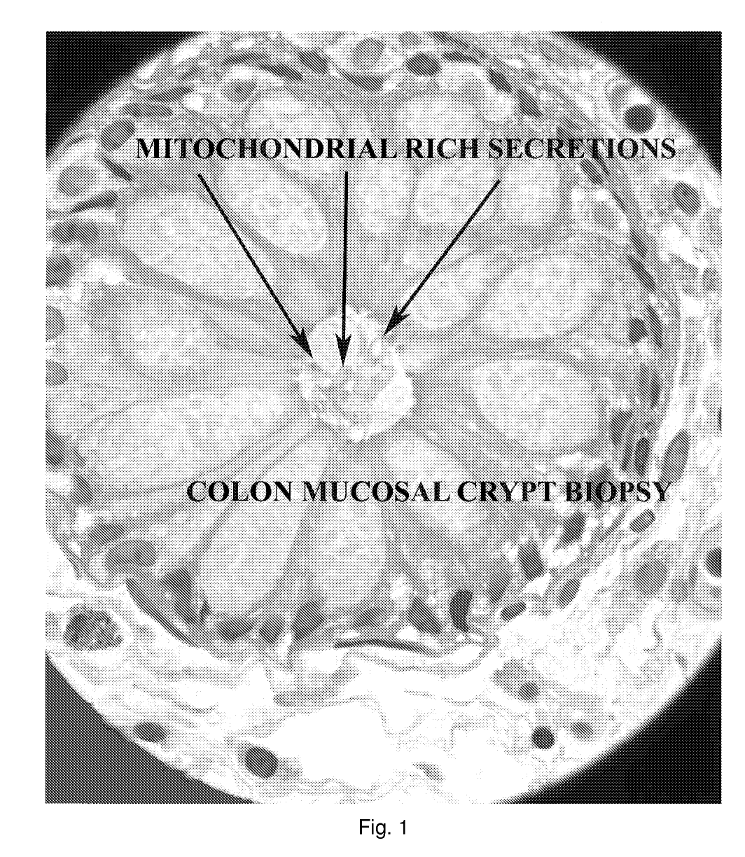

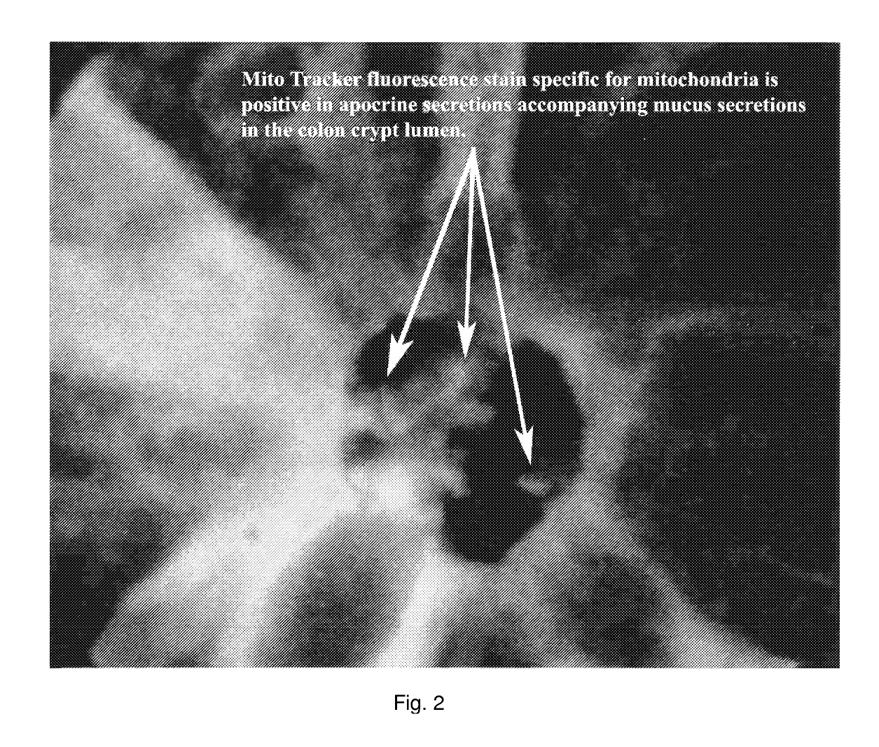

Applicant has discovered that humans and other life forms secrete mitochondria into the gastrointestinal tract. Such secretions of mitochondria occur primarily in the colon, but there may also be similar secretions into the small bowel and the stomach. Evidence that such mitochondria secretions into the gastrointestinal tract occur is shown in FIGS. 1-2. These Figures are pictures taken using light microscopy for which samples were prepared using the methods disclosed in U.S. Provisional Patent Application Ser. No. 60/670,119 filed on Apr. 11, 2005 entitled “Compositions and Methods for Preparing Specimens for Microscopic Analysis” (which provisional patent application is incorporated herein by reference). Specifically, FIG. 1 shows a stained mitochondria rich secretion from colon mucosal crypt biopsy in which the mitochondria rich secretion is indicated with arrows. Likewise, FIG. 2 shows mitochondria in apocrine secretions accompanying mucus secretions in the colon crypt lumen, wherein the presence of mitochondria is verified through the use of a mitochondria-specific Mito Tracker fluorescent stains.

The fact that mitochondria are secreted in the gastrointestinal tract provides a basis for the method of the present invention. Specifically, the present method involves using mass spectroscopy or similar analytical technique to analyze these gastrointestinal secretions that contain mitochondria and accompanying cytoplasmic secretions to obtain a proteomic pattern. The results of this mass spectroscopy will provide doctors with information about the proteome of the mitochondria in the gastrointestinal secretions, which will provide additional information regarding the patient's chronic diarrhea condition.

Specifically, the present method includes the step of obtaining a sample of gastrointestinal secretions that contain mitochondria. Generally, the sample of gastrointestinal secretions will usually be obtained while the patient is undergoing endoscopy, although other ways of obtaining the sample of gastrointestinal secretions may also be used. Common techniques known to those of skill in the art will be used to obtain these samples during the endoscopic procedures

Once the sample of gastrointestinal secretions has been obtained, this sample will generally be prepared for analysis using an analytical technique, including, but not limited to mass spectroscopy, gas chromatography-mass spectroscopy (GC-MS), GC-MS/MS or other chromatography techniques, biochemical analysis, tissue staining techniques such as antibody staining or fluorescence staining techniques. Sample preparation for the mass spectroscopic analysis may be done via commonly used methods, some of which are described in the article by David K. Crockett, et al. entitled “Identification of proteins from formalin-fixed paraffin-embedded cells by LC-MS/MS,” which article is incorporated herein by reference. While this article relates to recovering the tissue specimen from a paraffin block, it is within the level of skill in the art to prepare tissue specimens for use with mass spectroscopy, GC-MS/MS, and other analytical methods.

As noted above, the prepared sample will then be analyzed by mass spectroscopy, GC-MS/MS, or other appropriate analytical method. As is known in the art, conducting a mass spectrographic analysis on the sample will produce a mass chromatogram proteomic pattern with peaks that correspond to the various compounds, proteins, bio-molecules, etc. that are found in the sample. More specifically, the peaks include information about the proteome of mitochondria and cytoplasm in the gastrointestinal secretions. The proteomic pattern will be analyzed and compared to a proteomic pattern profile to provide information regarding the patient's chronic diarrhea condition.

The proteomic pattern profile database is created by taking similar mass spectroscopic proteomic patterns of a variety of patients, including those patients that are known to have chronic diarrhea and those patients who do not have such a condition.

By comparing the patient's proteomic pattern with the proteomic pattern profiles from the database, particular clues and information may be obtained. For example, the proteomic pattern of persons that suffer from chronic diarrhea will likely have one or more particular peaks in common. This peak or peaks will represent a particular compound, protein, biomolecule, etc. and will likely be the cause of the patient's diarrhea condition. Accordingly, the doctor will then be able to isolate and determine the identity of this compound, and more importantly, find ways to treat the defect that is being caused by this compound.

In other cases, comparing the proteomic pattern from the patient to the database may indicate that some patients suffering from chronic diarrhea lack a particular compound or protein that is present in “normal” patients—i.e., the proteomic patterns of these patients will lack a particular peak. Again, in such cases, the doctor will then know that the likely cause of the patient's diarrhea condition is the deficiency in this particular compound. Accordingly, the doctor can then treat the problem by adding this compound to the patient's system (via dietary supplements, injections, etc.).

Thus, by obtaining and analyzing mass spectroscopic proteomic pattern results from a particular patient, and then comparing these results with those of a known proteomic pattern profile database, a doctor will be able to diagnose chronic diarrhea.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method for obtaining information about a chronic diarrhea condition in a patient, the method comprising:
obtaining from the patient a sample of a gastrointestinal secretion that contains mitochondria;
performing mass spectroscopy on the sample to obtain a proteomic pattern; and
comparing the proteomic pattern of the sample with a proteomic pattern profile database to determine differences and similarities of the proteomic pattern to the proteomic pattern profile database.

2. A method as in claim 1 wherein the sample of gastrointestinal secretions further contains cytoplasmic secretions.

3. A method as in claim 1 wherein the comparing step includes analysis by adaptive artificial intelligence bioinformatics.

4. A method as in claim 1 wherein the mass spectroscopy utilizes surface-enhanced laser desorption ionization time-of-flight (SELDI-TOF).

5. A method as in claim 1 wherein the mass spectroscopy comprises matrix-assisted laser desorption ionization time-of-flight (MALDI-TOF) mass spectroscopy.

6. A method as in claim 1 wherein the mass spectroscopy is gas chromatography-mass spectroscopy (GC-MS).

7. A method as in claim 1 wherein the mass spectroscopy is gas chromatography-mass spectroscopy/mass spectroscopy (GC-MS/MS).

8. A method as in claim 1 wherein the sample of gastrointestinal secretions is obtained from the patient during endoscopy.

9. A method as in claim 1 wherein the database of proteomic pattern profiles includes proteomic pattern profiles from patients that do not have chronic diarrhea.

10. A method as in claim 1 wherein the database of proteomic pattern profiles includes proteomic pattern profiles from patients that do have chronic diarrhea or other gastrointestinal disorders.

11. A method as in claim 1 further comprising the step of using the differences and similarities of the proteomic pattern to obtain a possible diagnosis of chronic diarrhea.

12. A method as in claim 1 further comprising the step of using the differences and similarities of the proteomic pattern to determine that the patient lacks a particular compound or protein in the gastrointestinal secretion compared to patients that do not have chronic diarrhea.

13. A method as in claim 1 further comprising the step of using the differences and similarities of the proteomic pattern to determine that the patient possesses a different compound or protein in the gastrointestinal secretion compared to patients that do not have chronic diarrhea.
